(19) United States
(12) Patent Application Publication
(10) Pub. No.: US 2005/0048055 A1
(43) Pub. Date: Mar. 3, 2005

(54) Use of CD40 engagement to alter T cell receptor usage

(75) Inventors: Martha K. Newell, Colorado Springs, CO (US); Evan Newell, Colorado Springs, CO (US); David Wagner, Denver, CO (US)

Correspondence Address: Helen C. Lockhart, Ph.D.;Wolf, Greenfield & Sacks, P.C., 600 Atlantic Avenue, Boston, MA, 02210, US

(73) Assignees: University of Vermont and State Agricultural College, Burlington, VT (US); National Jewish Medical and Research Center, Denver, CO (US)

(21) Appl. No.: 10/939,652

(22) Filed: Sep. 13, 2004

Related U.S. Application Data
(63) Continuation of application No. 09/470,494, filed on Dec. 22, 1999, now abandoned.
(60) Provisional application No. 60/114,106, filed on Dec. 29, 1998.

(51) Int. Cl. A61K 39/395; C12N 5/08

(52) U.S. Cl. 424/144.1; 435/372

(57) ABSTRACT

The invention relates to methods for altering and enhancing the immune response toward an antigen. More specifically, the present invention relates to methods of using CD40 engagement on T cells to induce T cell receptor gene rearrangement and enhance T cell affinity for a particular antigen. The invention also relates to methods for promoting developmental maturation of an immature cell of the T cell lineage.

### RELATED APPLICATIONS

This application claims priority under 35 USC §119(e) from U.S. Provisional Patent Application Ser. No. 60/114,106, filed on Dec. 29, 1998, entitled USE OF CD40 ENGAGEMENT TO ALTER T CELL RECEPTOR USAGE. The contents of the provisional application are hereby expressly incorporated by reference.

### GOVERNMENT SUPPORT

This work was funded in part by grant number AI-33470 from the National Institutes of Health. Accordingly, the United States Government may have certain rights to this invention.

### FIELD OF THE INVENTION

This invention relates to methods for altering the immune response of a mammal toward an antigen. More specifically, the present invention relates to methods of using CD40 engagement on T cells to induce T cell receptor gene rearrangement and enhance T cell affinity for a particular antigen, and to promote maturation of a T cell.

### BACKGROUND OF THE INVENTION

A characteristic of the immune system is the specific recognition of antigens. This includes the ability to discriminate between self and non-self antigens and a memory-like potential that enables a fast and specific reaction to previously encountered antigens. The vertebrate immune system reacts to foreign antigens with a cascade of molecular and cellular events that ultimately results in the humoral and cell-mediated immune response.

The major pathway of the immune defense involving antigen-specific recognition commences with the trapping of the antigen by antigen presenting cells (APCs), such as dendritic cells or macrophages, and the subsequent migration of these cells to lymphoid organs (e.g., thymus). There, the APCs present antigen to subclasses of T cells classified as mature T helper cells. Upon specific recognition of the presented antigen, the mature T helper cells can be triggered to become activated T helper cells. The activated T helper cells regulate both the humoral immune response by inducing the differentiation of mature B cells to antibody producing plasma cells and the cell-mediated immune response by activation of mature cytotoxic T cells.

T lymphocytes recognize antigen in the context of the Major Histocompatibility Complex (MHC) molecules by means of the T cell receptor (TCR) expressed on their cell surface. The TCR is a disulfide linked heterodimer noncovalently associated with the CD3 complex (Allison, J. P., et al., Ann. Rev. Immunol., 1987, 5:503). Most T cells carry TCRs consisting of α and β glycoproteins. T cells use mechanisms to generate diversity in their receptor molecules similar to those operating in B cells (Kronenberg, M., et al., Ann. Rev. Immunol., 1986, 4:529; Tonegawa S., Nature, 1983, 302:575). Like the immunoglobulin (Ig) genes, the TCR genes are composed of segments which rearrange during T cell development. TCR and Ig polypeptides consist of amino terminal variable and carboxy terminal constant regions. The variable region is responsible for the specific recognition of antigen, whereas the C region functions in membrane anchoring and in transmitting of the signal that the receptor is occupied, from the outside to the inside of the cell. The variable region of the Ig heavy chain and the TCR β chain is encoded by three gene segments, the variable (V), diversity (D) and joining (J) segments. The Ig light chain and the TCR α chain contain variable regions encoded by V and J segments only.

The V, D and J segments are present in multiple copies in germline DNA. The diversity in the variable region is generated by random joining of one member of each segment family. Fusion of gene segments is accompanied by insertion of several nucleotides. This N-region insertion largely contributes to the diversity, particularly of the TCR variable regions (Davis and Bjorkman, Nature, 1986, 334:395), but also implies that variable gene segments are often not functionally rearranged. The rearrangement of gene segments generally occurs at both alleles. However, T and B cells express only one TCR or Ig respectively and two functionally rearranged genes within one cell have never been found. This phenomenon is known as allelic exclusion.

During thymocyte differentiation and development, thymocytes progress through a series of stages hallmarked by expression of cell surface molecules including CD4, CD8, TCR, and CD3. At early developmental stages thymocytes are multi-negative for expression of these molecules and during this developmental stage, the RAG-1 and RAG-2 gene products, which are necessary for TCR gene rearrangement events, become activated and rearrangement of the TCRβ gene occurs (Malissen, M, et al., Immunology Today, 1992, 13: 315-322). Only one allele of the TCRβ gene is expressed while the other allele is shut allowing TCR engagement (Lucas, B and Germain, R N, Immunity, 1996, 5: 461-477). From this population, both the CD4⁺8^{lo} and CD4^{lo}8⁺ sub-populations are generated (Lucas, B and Germain, R N, Immunity, 1996, 5: 461-477). CD4⁺8^{lo} cells give rise to double positive (DP) cells as well as both single positive (SP) thymocyte populations. The CD4^{lo}8⁺ population gives rise only to the CD8 SP population (Suzuki, H, et al., Immunity, 1995, 2: 413-425), and therefore is considered a more mature population than the CD4⁺8^{lo} population. The CD4^{lo}8^{lo}, CD69⁺ sub-population is most likely an intermediate population during CD4/CD8 lineage commitment.

Two different types of T cells are involved in antigen recognition within the MHC context. Mature T helper cells (CD4⁺8^{lo}) recognize antigen in the context of class II MHC molecules, whereas cytotoxic T cells (CD4^{lo}8⁺) recognize antigen in the context of class I MHC determinants (Swain, S. L., Immun. Rev., 1983, 74:129-142; Dialynas, P. D., et al., Immun. Rev., 1983, 74:29-56).

The CD40 surface molecule is a 277-amino-acid glycoprotein reported in the art to be expressed on B lymphocytes, epithelial cells and some carcinoma cell lines. Monoclonal antibodies against CD40 mediate a variety of effects on B lymphocytes, including induction of intercellular adhesion, short- and long-term proliferation, immunoglobulin gene rearrangement and immunoglobulin class switching events. CD40-Ligand (the natural CD40 binding partner) is reportedly expressed on the cell surface of activated T cells and mediates B-cell proliferation in the absence of co-stimulus, as well as IgE production in the presence of interleukin-4 (IL-4).

Neither the expression or function of CD40 on peripheral T cells, splenic T cells and thymocytes, however, has been determined.

### SUMMARY OF THE INVENTION

The invention, in one important part, provides methods for altering the specificity of T cells toward an antigen. More specifically, the present invention relates to methods of using CD40 engagement on thymocytes (or T cells) to induce T cell receptor gene rearrangement thus altering and enhancing T cell specificity toward an antigen. In another aspect, the invention provides methods for promoting maturation of thymocytes using CD40 engagement on thymocytes.

Surprisingly, according to the invention, it has been discovered that CD40 is expressed on the surface of thymocytes and that engagement of thymocyte-CD40 with a CD40 binding agent induces T cell receptor gene rearrangement in the thymocyte. Also surprisingly it has been discovered that developmentally mature T cells undergo rearrangement and alter their specificity according to the methods of the invention. Additionally, it has been discovered that engagement of an immature thymocyte expressing CD40 with a CD40 binding agent leads to developmental maturation of the thymocyte.

According to one aspect of the invention, a method for inducing T cell receptor gene rearrangement, is provided. The method involves contacting a T cell with a CD40-binding agent that binds CD40 in an amount sufficient to induce T cell receptor gene rearrangement in the T cell. Preferably the T cell is an isolated T cell. In certain embodiments, the T cell is free of an exogenous CD40 ligand encoding nucleic acid. In other embodiments, the T cell is present in a lymphocyte population enriched for T cells. In preferred embodiments, the lymphocyte population enriched for T cells is further enriched for T cells by selectively eliminating B cells. Various embodiments are provided, wherein the T cell-enriched lymphocyte population contains at least 50%, at least 75%, at least 90%, or at least 95% T cells. In certain other embodiments, contacting of the T cell with a CD40-binding agent that binds CD40 occurs in vitro. Other embodiments are provided, wherein contacting of the T cell with a CD40-binding agent that binds CD40 occurs ex vivo. In certain embodiments, the T cell may also be derived from an in vitro culture of hematopoietic cells.

In any of the foregoing embodiments, the CD40-binding agent preferably comprises at least two agents: i) a first agent that binds a first CD40 receptor, and ii) a second crosslinking agent that crosslinks the first agent to at least a second receptor that includes a second CD40 receptor and/or a T cell receptor. In some embodiments, the first agent that binds a first CD40 receptor includes a CD40 ligand and/or an anti-CD40 antibody. In other embodiments, the second crosslinking agent that crosslinks the first agent to the second receptor includes a CD40 ligand, an anti-CD40 antibody and/or an antigen. In preferred embodiments, the CD40 ligand is the polypeptide of SEQ ID NO:2 or a fragment thereof.

In any of the foregoing embodiments, T cells of a CD69⁺TCR⁺, CD4^{lo}CD8^{lo}CD69⁺ TCR⁺, CD4^{lo}CD8^{hi}CD69⁺TCR⁺, and/or CD4^{hi}CD8^{lo}CD69⁺TCR⁺ phenotype (mature) are particularly useful according to the invention.

Various embodiments are provided, where a co-stimulatory agent may be administered with the CD40 binding agent. The co-stimulatory agent includes a co-stimulatory molecule and a cytokine. Co-stimulatory molecules include, but are not limited to, TSA-1, CD2, CD5, CD24, CD28, CD49a, CD80, CD81 and CD86. Cytokines include, but are not limited to, IL-2 and IL-4.

According to another aspect of the invention, a method for promoting T cell maturation, is provided. The method involves contacting an immature T cell with a CD40-binding agent that binds CD40 in an amount sufficient to promote maturation of the immature T cell. Preferably the T cell is an isolated T cell. In certain embodiments, the T cell is free of an exogenous CD40 ligand encoding nucleic acid. In other embodiments, the T cell is present in a lymphocyte population enriched for T cells. In preferred embodiments, the lymphocyte population enriched for T cells is further enriched for T cells by selectively eliminating B cells. Various embodiments are provided, wherein the T cell-enriched lymphocyte population contains at least 50%, at least 75%, at least 90%, or at least 95% T cells. In certain other embodiments, contacting of the T cell with a CD40-binding agent that binds CD40 occurs in vitro. Other embodiments are provided, wherein contacting of the T cell with a CD40-binding agent that binds CD40 occurs ex vivo. In certain embodiments, the T cell may also be derived from an in vitro culture of hematopoietic cells.

In any of the foregoing embodiments, the CD40-binding agent preferably comprises at least two agents: i) a first agent that binds a first CD40 receptor, and ii) a second crosslinking agent that crosslinks the first agent to at least a second receptor that includes a second CD40 receptor and/or a T cell receptor. In some embodiments, the first agent that binds a first CD40 receptor includes a CD40 ligand and/or an anti-CD40 antibody. In other embodiments, the second crosslinking agent that crosslinks the first agent to the second receptor includes a CD40 ligand, an anti-CD40 antibody and/or an antigen. In preferred embodiments, the CD40 ligand is the polypeptide of SEQ ID NO:2 or a fragment thereof.

In any of the foregoing embodiments, T cells of a CD69⁻TCR^{lo}, CD4⁺CD8⁺TCR^{lo}, and/or CD117⁺TCR^{lo} phenotype are particularly useful according to the invention.

Various embodiments are provided, where a co-stimulatory agent may be administered with the CD40 binding agent. The co-stimulatory agent includes a co-stimulatory molecule and a cytokine. Co-stimulatory molecules include, but are not limited to, TSA-1, CD2, CD5, CD24, CD28, CD49a, CD80, CD81 and CD86. Cytokines include, but are not limited to, IL-2 and IL-4.

According to still another aspect of the invention, a method for inhibiting T cell receptor gene rearrangement, is provided. The method involves contacting a T cell expressing CD40 with an agent that inhibits CD40-induced T cell receptor rearrangement. Preferably the T cell is an isolated T cell. In certain embodiments, an agent that inhibits CD40-induced T cell receptor rearrangement includes an anti-CD40 ligand antibody, a soluble CD40 ligand antagonist, a NF-κB inhibitor, and/or any combinations thereof. Various embodiments are provided, wherein the CD40-binding agent, cell populations (both mature and immature T cells) and numbers, and co-administration of other co-stimulatory molecules have one or more of the preferred characteristics as described above. Such methods can be used to inhibit T cell affinity maturation towards a specific antigen(s), and in particular a self-antigen(s) (as in an autoimmune disease). In certain embodiments, the autoimmune disease includes rheumatoid arthritis, uveitis, insulin-dependent diabetes mellitus, hemolytic anemias, rheumatic fever, Crohn's disease, Guillain-Barre syndrome, psoriasis, thyroiditis, Graves' disease, myasthenia gravis, glomerulonephritis, autoimmune hepatitis, systemic lupus erythematosus. In further embodiments, the subject has multiple sclerosis, an abscess, a transplant, an implant, atherosclerosis, and/or myocarditis.

According to a further aspect of the invention, a method for inducing T cell reactivity toward an antigen, ex vivo and/or in vitro is provided. The method involves introducing an amount of T cells and an amount of antigen presenting cells into a culture vessel, and co-culturing the T cells and the antigen presenting cells in the presence of a CD40-binding agent that binds CD40 in an amount sufficient to induce T cell receptor gene rearrangement in the T cells, and at least one antigen, under conditions sufficient to induce the formation of T cells having specificity for the at least one antigen. Preferably the T cell is an isolated T cell. Various embodiments are provided, wherein the CD40-binding agent, mature T cell populations and co-administration of other co-stimulatory molecules have one or more of the preferred characteristics as described above.

In yet another aspect, the invention provides a method for inhibiting environmental stress-induced T cell-death. The method involves contacting a T cell naturally expressing CD40 (i.e. nonCD40-transfected), under environmental stress otherwise sufficient to induce cell-death, with a CD40-binding agent that binds CD40 and induces T cell receptor gene rearrangement in an amount sufficient to inhibit death of the T cell expressing CD40 which otherwise would result from the environmental stress. Preferably the T cell is an isolated T cell. In certain embodiments the environmental stress includes chemical stress, physical stress, oxidative stress, and/or γ-irradiation. Various other embodiments are provided, wherein the CD40-binding agent, cell populations (both mature and immature T cells) and numbers have one or more of the preferred characteristics as described above.

According to yet another aspect, a method for enhancing environmental stress-induced T cell-death, is provided. The method involves contacting a T cell naturally expressing CD40 (i.e. nonCD40-transfected), with a CD40-binding agent that binds CD40 in an amount sufficient to induce T cell receptor gene rearrangement, and subjecting the CD40-binding agent bound T cell to an environmental stress sufficient to induce cell-death. Preferably the T cell is an isolated T cell. In certain embodiments the environmental stress includes chemical stress, physical stress, oxidative stress, and/or γ-irradiation. In preferred embodiments, a T cell includes a cancerous T cell or a self-reactive T cell, and the environmental stress is a chemotherapeutic agent or a chemical agent. Various other embodiments are provided, wherein the CD40-binding agent, cell populations (both mature and immature T cells) and numbers, have one or more of the preferred characteristics as described above.

These and other aspects of the invention are described in greater detail below. Each of the limitations of the invention can encompass various embodiments of the invention. It is, therefore, anticipated that each of the limitations of the invention, involving any one element or combinations of elements can be included in each aspect of the invention.

These and other aspects of the invention, as well as various advantages and utilities, will be more apparent with reference to the detailed description of the preferred embodiments.

### BRIEF DESCRIPTION OF THE DRAWINGS

FIG. 1 shows the expression of CD40 on BALB/c thymocytes. Histograms representing CD40 expression in the gated, thymic CD4/CD8 subpopulations of FIG. 1A are shown in FIG. 1B, light line. Dark solid line represents a rat IgG isotype control.

FIG. 2 shows the effects of CD40 signaling on thymocyte T Cell Receptor (TCR) expression. TCR expression of the gated, thymic CD4/CD8 subpopulations of FIG. 2A are shown for the CD4⁺8⁺ sub-population (FIG. 2B), CD4⁺ thymocyte sub-population (FIG. 2C), CD8⁺ sub-population (FIG. 2D) and CD4⁻8⁻ sub-population (FIG. 2E). Dashed line represents untreated, and solid line represents anti-CD40 treated thymocytes. The histogram in FIG. 2F represents a CD4⁺8⁺ sub-population of CD3-depleted, CD40L fusion protein treated (solid line) of thymocytes.

FIG. 3 shows TCRVα8 (FIG. 3B) expression on untreated (FIG. 3B-dashed line) and CD40 crosslinked (FIG. 3B-solid line) of the CD4⁺8⁺ sub-population of thymocytes of FIG. 3A.

FIG. 4 shows that CD40 signals alter TCRVα11 expression on thymocytes.

FIG. 4 shows untreated (FIG. 4A) and anti-CD40 treated (FIG. 4B) thymocytes.

FIG. 5 shows expression of CD69 and TCRαβ on CD4^{lo}CD8^{lo} untreated (FIG. 5A) and CD40L-trimeric fusion protein crosslinked (FIG. 5B) thymocytes using Contour plots.

### BRIEF DESCRIPTION OF THE SEQUENCES

SEQ ID NO:1 is the nucleotide sequence of the human CD40-Ligand cDNA, Genbank Acc. No. L07414.

SEQ ID NO:2 is the polypeptide sequence of the human CD40-Ligand cDNA, encoded for by the nucleic acid of SEQ ID NO:1.

### DETAILED DESCRIPTION OF THE INVENTION

The invention in one aspect involves the unexpected discovery that CD40 is expressed on the surface of thymocytes and that engagement of thymocyte-CD40 with a CD40-binding agent induces T cell receptor gene rearrangement in the thymocyte. Thus, the present invention, and in contrast to what has been previously believed in the art, is useful in changing the antigen specificity of a peripheral thymocyte (i.e. a “mature” T cell, e.g., a CD4^{lo}CD8^{lo}CD69⁺TCR⁺, or a T cell committed to a specific antigen).

According to one aspect of the invention, a method for inducing T cell receptor gene rearrangement in vitro and/or ex vivo, is provided. By “ex vivo” it is meant that cells have been isolated from a subject, are temporarily cultured and manipulated in vitro, and returned to the subject. As used herein, a subject is a human, non-human primate, cow, horse, pig, sheep, goat, dog, cat or rodent. In all embodiments human subjects are preferred. Alternatively, the cells may be obtained from an in vitro culture of hematopoietic cells (hematopoietic stem and progenitor cells), cultured toward a T cell lineage and manipulated in vitro, and then introduced into a subject. Such in vitro hematopoietic progenitor cell cultures are well known in the art and examples include the methods and systems described in U.S. Pat. No. 5,635,386, entitled “Methods for regulating the specific lineages of cells produced in a human hematopoietic cell culture”, issued to Palsson et al., and U.S. Pat. No. 5,646,043, entitled “Methods for the ex vivo replication of human stem cells and/or expansion of human progenitor cells”, issued to Emerson, et al.

Induction of T cell receptor gene rearrangement in a T cell involves contacting a T cell with a CD40-binding agent that binds CD40 in an amount sufficient to induce T cell receptor gene rearrangement in the T cell.

The T cell treated according to the methods of the present invention is preferably an isolated T cell. An isolated T cell as used herein is a cell within a lymphocyte population that is enriched for T cells by selectively eliminating B cells, although a small number of B cells may be present. Methods for T cell enrichment are described in more detail below (see under isolation of peripheral blood or monocytes). Various embodiments are provided, wherein the T cell-enriched lymphocyte population contains at least 50%, at least 75%, at least 90%, or at least 95% T cells. Also preferred is that the T cell is free of an exogenous CD40 ligand encoding nucleic acid.

According to the invention, a CD40-binding agent that binds one or more receptors on the surface of a T cell is required to induce T cell receptor gene rearrangement in a mature T cell, and/or induce the maturation of an immature T cell (see discussion below). Methods for detecting any of the foregoing effects are described in the Examples. A “CD40-binding agent” preferably is composed of at least two agents: i) a first agent that binds a first CD40 receptor on the surface of a T cell, and ii) a second crosslinking agent that crosslinks the first agent to at least a second receptor, the second receptor including a second CD40 receptor and/or a T cell receptor. Although the second receptor may be present on the surface of the same T cell as the first receptor, the second receptor may also be present on the surface of another T cell. Simultaneous binding of two T cell surface receptors on the same cell, one of which is the first CD40 receptor, may also exert any of the foregoing effects.

A “first agent that binds a first CD40 receptor” as used herein, is any compound known in the art to bind to a CD40 receptor and includes, for example, a CD40 ligand and/or an anti-CD40 antibody. A “second crosslinking agent that crosslinks the first agent to a second receptor” as used herein, is any compound known in the art to bind to and crosslink a compound to a CD40- and/or T cell-receptor, and includes, for example, a CD40 ligand, an anti-CD40 antibody and/or an antigen. CD40-binding agents thus include molecules that crosslink two or more cell surface receptors on a T cell, one of which is the first CD40 receptor, the other a second CD40 receptor or a T cell receptor. Examples of CD40-binding agents thus include CD40 ligand multimers, immobilized CD40 ligand monomers, anti-CD40 antibodies that are conjugated through, for example, biotin/avidin(streptavidin) bonds, etc. A preferred CD40 ligand multimer is a CD40 trimer. By “immobilized” it is meant that the agent is attached to a solid support and it is thus in a nonsoluble form. The chemistry for attaching linker moieties for connecting two agents is well known and commonly used in the art (see also later discussion on immobilization). Anti-CD40 antibody and other agents described herein are commercially available (e.g., Pharmingen) (see Examples).

A preferred CD40 ligand is the polypeptide of SEQ ID NO:2 or an active fragment thereof. By “active fragment” it is meant to include a fragment of the polypeptide that maintains the activity of the polypeptide, i.e. it binds a CD40 receptor on the surface of a T cell and exerts any of the effects the full-length CD40 polypeptide exerts according to the invention. The polypeptide of SEQ ID NO:2 may be encoded for by the nucleic acid of SEQ ID NO:1. The CD40 ligand, however, alternatively may also be a polypeptide encoded for by other isolated nucleic acid molecules that code for a CD40 ligand polypeptide and include: (a) nucleic acid molecules which hybridize under stringent conditions to a molecule consisting of a nucleic acid of SEQ ID NO:1 and which code for a CD40 ligand polypeptide, (b) deletions, additions and substitutions of (a) which code for a respective CD40 ligand polypeptide, (c) nucleic acid molecules that differ from the nucleic acid molecules of (a) or (b) in codon sequence due to the degeneracy of the genetic code, and (d) complements of (a), (b) or (c). Homologs and alleles of SEQ ID NO:1 may also encode for a CD40 ligand polypeptide. In general homologs and alleles typically will share at least 40% nucleotide identity to SEQ ID NO:1 and/or at least 50% amino acid identity to the CD40 ligand polypeptide encoded for by SEQ ID NO:1. The homology can be calculated using various, publicly available software tools developed by NCBI (Bethesda, Md.) that can be obtained through the Internet (ftp:/ncbi.nlm.nih.gov/pub/). Exemplary tools include the BLAST system available at http://www.ncbi.nlm.nih.gov/. Pairwise and ClustalW alignments (BLOSUM30 matrix setting) as well as Kyte-Doolittle hydropathic analysis can be obtained using the MacVector sequence analysis software (Oxford Molecular Group).

The CD40-binding agent is added to a T cell culture in an amount sufficient to induce T cell receptor gene rearrangement in the T cell. The “amount sufficient” to induce T cell receptor gene rearrangement in the T cell is an amount of the CD40-binding agent that can be easily determined by a person skilled in the art, and can vary depending upon the original number of T cells seeded and the culture conditions used. The amounts of T cells initially seeded in the culture vessel may vary according to the needs of the experiment. The ideal amounts can be easily determined by a person skilled in the art in accordance with needs. The culture conditions used refer to a combination of conditions known in the art (e.g., temperature, CO₂ and O₂ content, nutritive media, type of culture vessel, time-length, etc.).

For the ex vivo part of the invention, mononuclear cells to be processed according to the invention can be obtained from subjects. The subjects may be afflicted with a malignant tumor or an infectious disease such as hepatitis B. Peripheral blood or a mononuclear cell-enriched population of cells (obtained using known methods, e.g., apheresis) is taken from a subject, and a portion of the sample is mixed with an anticoagulant, e.g., heparin, sodium citrate, ethylenediaminetetraacetic acid, sodium oxalate. The blood-anticoagulant mixture then is diluted in a physiologically acceptable solution such as sodium chloride or phosphate buffered solution. Mononuclear cells are recovered by layering the blood-anticoagulant composition onto a centrifugation separation medium such as Ficoll-Hypaque (Pharmacia Corporation) or Lymphocyte Separation Medium (Litton Bionetics Corporation). The layered mixture then is centrifuged, and the interface containing the mononuclear cells is collected and washed. The concentration of mononuclear cells can be in the range of about 0.5-5.0×10⁶ cells/ml, preferably 1.0-2.0×10⁶ cells/ml. Although any standard tissue culture medium can be utilized in the process of this invention, the cells are preferably cultured in a complete medium consisting of RPMI 1640 (Gibco-BRL, Grand Island, N.Y.), supplemented with 2 mM L-Glutamine, streptomycin (100 mg/ml), penicillin (100 U/ml, and 5% heat-inactivated autologous plasma. Enriched monocyte preparations can be prepared by rosetting of PMBCs with AET-treated sheep red blood cells and removal of E-rosetting cells on Ficoll-Hypaque density gradients, followed by cold aggregation of monocytes as essentially described in Zupo et al. (Eur. J. Immunol., 1991, 21:351). T cells may be further purified from the PMBC preparations by depletion of monocytes, B cells and NK cells using Lympho-Kwik T (One Lambda, Los Angeles, Calif.) according to the manufacturer's protocol.

In certain embodiments, the T cell is a mature T cell. According to the present invention a “mature” T cell is a fully functional T cell, i.e. it has rearranged its T cell receptor and possesses the ability to exit the thymus. Examples of mature T cells include cells of a CD4^{lo}CD8^{lo}CD69⁺TCR⁺, a CD4^{lo}CD8^{hi}CD69⁺TCR⁺, and/or a CD4^{hi}CD8^{lo}CD69⁺TCR⁺ phenotype. A number of various other mature T cell phenotypes exist and the skilled artisan would be able to distinguish them from an immature cell, using phenotypic characteristics as well as functional assays well known in the art.

Proteins, peptides and other molecules including CD40-binding agents, can be immobilized on solid-phase matrices for use in accordance with the methods of the invention. The matrices may be agarose, beaded polymers, polystyrene/polypropylene plates or balls, porous glass or glass slides, and nitrocellulose or other membrane materials. Some supports can be activated for direct coupling to a ligand. Other supports are made with nucleophiles or other functional groups that can be linked to proteins or other ligands using cross-linkers.

Immobilization of the molecules of the invention to solid-supports can be accomplished using routine coupling chemistries. In general, the compounds of the invention are immobilized by including in the compounds an accessible first functional group (e.g., an alcohol group) and contacting the compound with a solid-support containing a complementary second functional group (e.g., carboxyl groups) under conditions and for a period of time sufficient to permit the first and the second functional groups to react with one another to form a covalent bond (e.g., ester bond). By “accessible” in reference to a functional group, it is meant that the functional group is in a form which is reactive and is not sterically precluded from reacting with the solid-support. Attachment can be direct or indirect (i.e., via a linker).

The present invention is also useful in promoting maturation of an immature T cell (naive thymocyte). The method involves contacting an immature T cell with a CD40-binding agent that binds CD40 in an amount sufficient to promote maturation of the immature T cell. The “amount sufficient” to promote maturation of an immature T cell is an amount of the CD40-binding agent that can be easily determined by a person skilled in the art, and can vary depending upon the initial cell-phenotype—i.e. maturation-stage of the T cell, the original number of T cells seeded and the culture conditions used. The amounts of T cells initially seeded in the culture vessel may vary according to the needs of the experiment. The ideal amounts can be easily determined by a person skilled in the art in accordance with needs. According to the invention, an immature T cell is a non-fully functional T cell that cannot exit the thymus. Examples of immature cells include cells of a CD4⁺CD8⁺TCR^{lo}, CD117⁺TCR^{lo}, etc., phenotype. A number of various other immature T cell phenotypes exist and the skilled artisan would be able to distinguish them from a mature cell, using phenotypic characteristics as well as functional assays well known in the art. According to another aspect of the invention, a method for inducing T cell reactivity toward an antigen, ex vivo and/or in vitro is provided. The method involves introducing an amount of T cells and an amount of antigen presenting cells into a culture vessel, and co-culturing the T cells and the antigen presenting cells in the presence of a CD40-binding agent that binds CD40 in an amount sufficient to induce T cell receptor gene rearrangement in the T cells, and at least one antigen, under conditions sufficient to induce the formation of T cells having specificity for the at least one antigen. One or more of the foregoing antigens can be used at the same time for incubation in a culture vessel. The foregoing conditions could easily be established by a person of ordinary skill in the art, without undue experimentation (see also Sprent J, et al., J Immunother, 1998, 21(3):181-187; Berridge M J, Crit Rev Immunol, 1997, 17(2):155-178; Owen M J, et al., Curr Opin Immunol, 1996, 8(2):191-198; Whitfield J F, et al., Mol Cell Biochem, 1979, 27(3):155-179; Fauci A S, et al., Ann Intern Med, 1983, 99(1):61-75). Antigen stimulation of T cells in the presence of APCs and a CD40-binding agent that binds CD40, induces T cell receptor gene rearrangement and an antigen specific response that can be measured using a proliferation assay or just by measuring IL-2 production (see discussion below). These cells can be detected by culturing T cells with antigen at an appropriate concentration (e.g., 0.1-1.0 μM tetanus toxoid) in the presence of APCs. If antigen specific T cells are present they can be detected using assays well known in the art such as radio-active assays or commercially available non-radioactive, ELISA based assays (e.g. Promega, Madison, Wis.).

Stimulation of T cells in the presence of APCs and a CD40-binding agent that binds CD40 may include co-stimulation with a co-stimulatory agent. Co-stimulatory agents include TSA-1, CD2, CD5, CD24, CD28, CD49a, CD80, CD81 and CD86, and cytokines such as IL-2 and IL-4. Co-stimulatory agents may also be used in lieu of APCs, provided that MHC class II molecules and anti-CD3 antibodies are co-administered with the co-stimulatory agent(s). Therefore, large numbers of antigen-specific mature T cells may be obtained. The present invention thus becomes useful in a wide range of applications, including pre-exposure vaccination of individuals with in vitro primed T cells, treatment of cancer subjects using tumor-targeted T cell immunotherapy, treatment of bone marrow transplant subjects (for whom opportunistic infections, such as CMV, are problematic and yet amenable to treatment with targeted T cells such as CMV-targeted cytotoxic lymphocytes), enhancement of conventional vaccination efficacy through T cell adjuvant therapy, treatment of outbreaks of emergent or re-emergent pathogens, etc. The antigen presenting cells include cells such as dendritic cells, monocytes/macrophages, Langerhans cells, Kupfer cells, microglia, alveolar macrophages, and methods for their isolation are well known in the art. The thymocytes as well as the antigen presenting cells may also be derived from hematopoietic progenitor cells in vitro.

As described above, antigens that can be used in accordance with the methods of the invention include antigens characteristic of pathogens and cancer antigens.

Antigens that are characteristic of tumor antigens typically will be derived from the cell surface, cytoplasm, nucleus, organelles and the like of cells of tumor tissue. Examples include antigens characteristic of tumor proteins, including proteins encoded by mutated oncogenes; viral proteins associated with tumors; and tumor mucins and glycolipids. Tumors include, but are not limited to, those from the following sites of cancer and types of cancer: lip, nasopharynx, pharynx and oral cavity, esophagus, stomach, colon, rectum, liver, gall bladder, biliary tree, pancreas, larynx, lung and bronchus, melanoma of skin, breast, cervix, uteri, uterus, ovary, bladder, kidney, brain and other parts of the nervous system, thyroid, prostate, testes, Hodgkin's disease, non-Hodgkin's lymphoma, multiple myeloma and leukemia. Viral proteins associated with tumors would be those from the classes of viruses noted above. Antigens characteristic of tumors may be proteins not usually expressed by a tumor precursor cell, or may be a protein which is normally expressed in a tumor precursor cell, but having a mutation characteristic of a tumor. An antigen characteristic of a tumor may be a mutant variant of the normal protein having an altered activity or subcellular distribution. Mutations of genes giving rise to tumor antigens, in addition to those specified above, may be in the coding region, 5′ or 3′ noncoding regions, or introns of a gene, and may be the result of point mutations, frameshifts, deletions, additions, duplications, chromosomal rearrangements and the like. One of ordinary skill in the art is familiar with the broad variety of alterations to normal gene structure and expression which gives rise to tumor antigens. Specific examples of tumor antigens include: proteins such as Ig-idiotype of B cell lymphoma, mutant cyclin-dependent kinase 4 of melanoma, Pmel-17 (gp100) of melanoma, MART-1 (Melan-A) of melanoma, p15 protein of melanoma, tyrosinase of melanoma, MAGE 1, 2 and 3 of melanoma, thyroid medullary, small cell lung cancer, colon and/or bronchial squamous cell cancer, BAGE of bladder, melanoma, breast, and squamous cell carcinoma, gp75 of melanoma, oncofetal antigen of melanoma; carbohydrate/lipids such as muc1 mucin of breast, pancreas, and ovarian cancer, GM2 and GD2 gangliosides of melanoma; oncogenes such as mutant p53 of carcinoma, mutant ras of colon cancer and HER-2/neu proto-oncogene of breast carcinoma; viral products such as human papilloma virus proteins of squamous cell cancers of cervix and esophagus. It is also contemplated that proteinaceous tumor antigens may be presented by HLA molecules as specific peptides derived from the whole protein. Metabolic processing of proteins to yield antigenic peptides is well known in the art; for example see U.S. Pat. No. 5,342,774 (Boon et al.).

Preferred tumor antigens of the invention include the Melonoma tumor antigens (e.g., MAGE protein family (MAGE-1, MAGE-2, MAGE-3); MART-1 (peptide 27-35); and gp100); and the Colon carcinoma antigens (e.g., peptides of the mutated APC gene product). Particularly preferred Melanoma tumor antigen sequences are those reported by Slingluff et al., in Curr. Opin. in Immunol., 1994, 6:733-740.

A variety of “culture vessels” can be used according to the present invention. Commercially available incubation vessels include stirring flasks (Corning, Inc., Corning, N.Y.), stirred tank reactors (Verax, Lebanon, N.H.), airlift reactors, suspension cell reactors, cell adsorption reactors and cell entrapment reactors, petri dishes, multiwell plates, flasks, bags and hollow fiber devices, Cellfoam (Cytomatrix, Woburn, Mass.), maxisorb plates (NUNC), and cell culture systems (e.g., Aastrom Cell Production System, see also U.S. Pat. No. 5,635,386, entitled “Methods for regulating the specific lineages of cells produced in a human hematopoietic cell culture”, issued to Palsson et al., and U.S. Pat. No. 5,646,043, entitled “Methods for the ex vivo replication of human stem cells and/or expansion of human progenitor cells”, issued to Emerson, et al.). In general, the cell cultures using the above-noted culture vessels are maintained in suspension by a variety of techniques including stirring, agitation or suspension by means of beads. In general, such vessels are formed of one or more of the following components: polystyrene, polypropylene, acrylic, nylon, and glass.

The insoluble matrices listed above do not themselves possess functional groups for the attachment of compounds of the invention, and must therefore be chemically modified, a process known as activation. For example, polystyrene can be activated chloromethylation of the phenyl residues (Pierce Chemical Company Catalog and Handbook; Combinatorial Peptide & Nonpeptide Libraries. A Handbook. V C H Weinheim Ed. Giuntha Jung—1996—Chapter 16 & 17) to yield chloromethyl polystyrene. Advantage can then be taken of the reactive benzylic chloride functional group to introduce carboxylate, amino, hydroxyl, maleimide, sulfhydryl, N-succinimidyl, and many other functional groups. The introduction of the functional groups then permits chemistries to be carried out which permit the covalent attachment of compounds of the invention either directly or through a linker spacer unit. The linking reactions require compatible functional groups on the matrix and the ligand or spacer-linker group which is or will be attached to the compound of the invention. For example, introduction of a carboxylate group on the matrix permits covalent coupling to free amino groups.

The chemistry leading to such coupling is well-known and described in many sources including in the catalogues of companies such as Pierce Chemical which sells both the matrices, activated and unactivated, and linker-spacer molecules. Other suppliers include, for example, Sigma, Novabiochem, among others. Methods for attaching ligands as described above for polystyrene but specific for the other matrices listed above are available, well known, and described in sources such as those described above and Immobilized Affinity Ligand Techniques. “All the ‘recipes’ for successful affinity matrix preparation”; Chemistry of Protein Conjugation and Cross-linking, by Shan S. Wong.

Avidin-Biotin chemistry provides another way of achieving the same end result, the attachment of the compounds of the invention to insoluble matrices. Biotin can easily be attached to a CD40-binding agent, for example, and the resulting conjugate will adhere with high affinity to avidin or streptavidin. A wide assortment of insolubilized derivatives of avidin and streptavidin are available commercially (Avidin-Biotin Chemistry: A Handbook—Developed by Pierce Technical Assistance experts).

According to still another aspect of the invention, a method for inhibiting T cell receptor gene rearrangement, is provided. The method involves contacting a T cell expressing CD40 with an agent that inhibits CD40-induced T cell receptor rearrangement. In certain embodiments, an agent that inhibits CD40-induced T cell receptor rearrangement includes an anti-CD40 ligand antibody, a fragment or derivative of an anti-CD40 ligand antibody, a soluble CD40 ligand antagonist, soluble forms of a fusion CD40 ligand protein, agents which disrupt or interfere with the CD40-CD40 ligand interaction, NF-κB inhibitor, and/or any combinations thereof. A “soluble CD40 ligand antagonist” refers to a soluble ligand that binds to CD40 on the surface of a lymphocyte and prevents the binding of the natural ligand (CD40 ligand) to CD40, resulting in the prevention of intracellular signal transduction leading to e.g., T cell receptor gene rearrangement. Non-stimulatory antagonistic monoclonal antibodies, for example, that can bind to human CD40 located on a cell surface are described in WO 94/01547. Additionally, CD40 signals are mediated through the nuclear factor NF-κB (Poe J., et al., J. Immunology, 1997, 159:846-52; Seetharaman R., et al., J. Immunology, 1999, 163:1577-1583). “NF-κB inhibitors” are therefore useful in inhibiting CD40 mediated signals and can be used alone or in combination with any of the foregoing agents that inhibit CD40-induced T cell receptor rearrangement. NF-κB inhibitors are well known in the art and include, but are not limited to, IκBα super-repressor, curcumin, phenylarsine oxide, SN-50, acrolein, ceramide, flavonoids (e.g., myricetin), and the like. A preferred NF-κB inhibitor according to the invention is the naturally occuring NF-κB inhibitor IκBα super-repressor (Boothby M, et al., J. Exp. Med., 1997, 185:1897-1907; Seetharaman R., et al., J. Immunology, 1999, 163:1577-1583). The IκBα super-repressor cDNA is preferably incorporated in an expression vector. A preferred such expression vector is an adenoviral vector (see, e.g., ADV IκBα S32A/S36E: Chu Z., et al., Proc Natl Acad Sci USA 1997, 94, 10057-10062). Inhibition of T cell receptor rearrangement results in inhibition of T cell affinity maturation towards a specific antigen(s). Such inhibition of T cell affinity maturation, especially toward a self-antigen(s), is desireable in a number of disorders, including autoimmune disease. “Autoimmune disease” as used herein, results when a subject's immune system attacks its own organs or tissues, producing a clinical condition associated with the destruction of that tissue, as exemplified by diseases such as rheumatoid arthritis, uveitis, insulin-dependent diabetes mellitus, hemolytic anemias, rheumatic fever, Crohn's disease, Guillain-Barre syndrome, psoriasis, thyroiditis, Graves' disease, myasthenia gravis, glomerulonephritis, autoimmune hepatitis, multiple sclerosis, systemic lupus erythematosus, etc.

Autoimmune disease may be caused by a genetic predisposition alone, by certain exogenous agents (e.g., viruses, bacteria, chemical agents, etc.), or both. Some forms of autoimmunity arise as the result of trauma to an area usually not exposed to lymphocytes, such as neural tissue or the lens of the eye. When the tissues in these areas become exposed to lymphocytes, their surface proteins can act as antigens and trigger the production of antibodies and cellular immune responses which then begin to destroy those tissues. Other autoimmune diseases develop after exposure of a subject to antigens which are antigenically similar to, that is cross-reactive with, the subject's own tissue. In rheumatic fever, for example, an antigen of the streptococcal bacterium, which causes rheumatic fever, is cross-reactive with parts of the human heart. The antibodies cannot differentiate between the bacterial antigens and the heart muscle antigens, consequently cells with either of those antigens can be destroyed.

Other autoimmune diseases, for example, insulin-dependent diabetes mellitus (involving the destruction of the insulin producing beta-cells of the islets of Langerhans), multiple sclerosis (involving the destruction of the conducting fibers of the nervous system) and rheumatoid arthritis (involving the destruction of the joint-lining tissue), are characterized as being the result of a mostly cell-mediated autoimmune response and appear to be due primarily to the action of T cells (See, Sinha et al., Science, 1990, 248:1380). Yet others, such as myesthenia gravis and systemic lupus erythematosus, are characterized as being the result of primarily a humoral autoimmune response. Nevertheless, inhibition of T cell receptor rearrangement involved in any of the foregoing conditions according to the invention, is beneficial to a subject (in need of such therapy) since inhibition of T cell affinity maturation towards a specific antigen(s) prevents escalation of the inflammatory response, protecting the specific site (e.g., tissue) involved, from “self-damage.” In preffered embodiments, the subject has rheumatoid arthritis, multiple sclerosis, or uveitis. In certain embodiments, the autoimmune disorder includes rheumatoid arthritis, uveitis, insulin-dependent diabetes mellitus, hemolytic anemias, rheumatic fever, Crohn's disease, Guillain-Barre syndrome, psoriasis, thyroiditis, Graves' disease, myasthenia gravis, glomerulonephritis, autoimmune hepatitis, systemic lupus erythematosus. In further embodiments, the subject has multiple sclerosis, an abscess, a transplant, an implant, atherosclerosis, and/or myocarditis.

In a further aspect, the invention provides a method for inhibiting environmental stress-induced cell-death of a T cell naturally expressing CD40 (i.e. nonCD40-transfected with a CD40 containing vector) and under an environmental stress otherwise sufficient to induce cell-death, by contacting the T cell with a CD40-binding agent that binds CD40 and increases T cell receptor gene rearrangement in an amount sufficient to inhibit death of the T cell which otherwise would result from the environmental stress. The ability of a CD40 binding agent to “inhibit death of the T cell” is assessed by a change in lifespan of the T cell. The lifespan of a cell under environmental stress is significantly shorter when compared to the lifespan of a cell under no such stress. This can be easily detected by placing a number of cells under a form of environmental stress and comparing their survival (numbers) to an identical number of cells free from any stress over a period of time. By “lifespan,” as used herein, it is meant to describe in terms of time the average life of a mammalian cell (e.g., a T cell) from its formation from a progenitor cell to its death. The average lifespan, for example, of a human red blood cell in circulation is on average 120 days. Any type of environmental stress (of enough severity) applied onto such cell is likely to reduce this average lifespan. The amount of the foregoing CD40-binding agent(s) of the invention sufficient to increase T cell receptor gene rearrangement in a T cell and inhibit cell-death, is the amount sufficient to extend the life of the mammalian cell under environmental stress beyond the time the cell would survive had it not come in contact with a CD40-binding agent of the invention, and toward comparable lifespan lengths of cells free from any environmental stress. The lifespan length of a cell under environmental stress can be easily determined by a person of ordinary skill in the art, and it will vary depending upon the cell type and its maturation stage, the cell numbers originally seeded, the culture conditions, the type of environmental stress used, etc. Such methods can be used to protect cells from environmental insults, such as increased temperatures (e.g., fever), physical trauma, oxidative, osmotic and chemical stress, UV and γ-irradiation. Thus, the term “inhibit death of T cell” as used herein, refers to the ability to increase the lifespan of a T cell relative to another T cell under similar conditions.

In another aspect, the invention provides a method for enhancing environmental stress-induced T cell-death. The method involves contacting a T cell naturally expressing CD40 (i.e. nonCD40-transfected), with a CD40-binding agent that binds CD40 in an amount sufficient to induce T cell receptor gene rearrangement and sensitize the cell to cell-death inducing stimuli (e.g., an environmental stress). It has been discovered, unexpectedly, that the CD40-binding agent bound T cell when subjected to a cell-death inducing stimulus (e.g., an environmental stress), Fas-mediated cell-death is enhanced. Such methods are useful in treating a variety of conditions including autoimmune disorders (by eliminating self-reactive T cells), and T cell lymhomas (by eliminating proliferative T cells). In certain embodiments, and particularly in the treatment of the foregoing conditions, the environmental stress is of a chemical nature. Preferred chemical agents used according to the invention are anti-cancer agents. Anti-cancer agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division), incorporated herein by reference and hereinafter referred to as “Calabresi and Chabner in G & G”. Suitable chemotherapeutic agents may have various mechanisms of action. The classes of suitable chemotherapeutic agents include (a) Alkylating Agents such as nitrogen mustard (e.g. mechlorethamine, cylophosphamide, ifosfamide, melphalan, chlorambucil), ethylenimines and methylmelamines (e.g. hexamethylmelamine, thiotepa), alkyl sulfonates (e.g. busulfan), nitrosoureas (e.g. carmustine which is also known as BCNU, lomustine which is also known as CCNU semustine which is also known as methyl-CCNU, chlorozoticin, streptozocin), and triazines (e.g. dicarbazine which is also known as DTIC); (b) Antimetabolites such as folic acid analogs (e.g. methotrexate), pyrimidine analogs (e.g. 5-fluorouracil floxuridine, cytarabine, and azauridine and its prodrug form azaribine), and purine analogs and related materials (e.g. 6-mercaptopurine, 6-thioguanine, pentostatin); (c) Natural Products such as the vinca alkaloids (e.g. vinblastine, Vincristine), epipodophylotoxins (e.g. etoposide, teniposide), antibiotics (e.g. dactinomycin which is also known as actinomycin-D, daunorubicin, doxorubicin, bleomycin, plicamycin, mitomycin, epirubicin, which is 4-epidoxorubicin, idarubicin which is 4-dimethoxydaunorubicin, and mitoxanthrone), enzymes (e.g. L-asparaginase), and biological response modifiers (e.g. Interferon alfa); (d) Miscellaneous Agents such as the platinum coordination complexes (e.g. cisplatin, carboplatin), substituted ureas (e.g. hydroxyurea), methylhydiazine derivatives (e.g. procarbazine), adreocortical suppressants (e.g. mitotane, aminoglutethimide) taxol; and (e) Hormones and Antagonists such as adrenocorticosteroids (e.g. prednisone or the like), progestins (e.g. hydroxyprogesterone caproate, medroxyprogesterone acetate, megestrol acetate), estrogens (e.g. diethyestilbestrol, ethinyl estradiol, and the like), antiestrogens (e.g. tamoxifen), andorgens (e.g. testosterone propionate, fluoxymesterone, and the like), antiandrogens (e.g. flutamide), and gonadotropin-releasing hormone analogs (e.g. leuprolide).

Other approved anti-cancer agents include: Acivicin; aclarubicin; acodazole hydrochloride; acronine; adozelesin; aldesleukin; altretamine; ambomycin; ametantrone acetate; amsacrine; anastrozole; anthramycin; asperlin; azacitidine; azetepa; azotomycin; batimastat; benzodepa; bicalutamide; bisantrene hydrochloride; bisnafide dimesylate; bizelesin; brequinar sodium; bropirimine; cactinomycin; calusterone; caracemide; carbetimer; carubicin hydrochloride; carzelesin; cedefingol; cirolemycin; cladribine; crisnatol mesylate; dacarbazine; decitabine; dexormaplatin; dezaguanine; dezaguanine mesylate; diaziquone; docetaxel; droloxifene; droloxifene citrate; dromostanolone propionate; duazomycin; edatrexate; eflomithine hydrochloride; elsamitrucin; enloplatin; enpromate; epipropidine; erbulozole; esorubicin hydrochloride; estramustine; estramustine phosphate sodium; etanidazole; etoprine; fadrozole hydrochloride; fazarabine; fenretinide; floxuridine; fludarabine phosphate; flurocitabine; fosquidone; fostriecin sodium; gemcitabine; gemcitabine hydrochloride; hydroxyurea; ilmofosine; interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; liarozole hydrochloride; lometrexol sodium; losoxantrone hydrochloride; masoprocol; maytansine; megestrol acetate; melengestrol acetate; menogaril; metoprine; meturedepa; mitindomide; mitocarcin; mitocromin; mitogillin; mitomalcin; mitosper; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plomestane; porfimer sodium; porfiromycin; prednimustine; puromycin; puromycin hydrochloride; pyrazofurin; riboprine; rogletimide; safingol; safingol hydrochloride; simtrazene; sparfosate sodium; sparsomycin; spirogermanium hydrochloride; spiromustine; spiroplatin; streptonigrin; sulofenur; talisomycin; tecogalan sodium; tegafur; teloxantrone hydrochloride; temoporfin; teroxirone; testolactone; thiamiprine; tiazofurin; tirapazamine; topotecan hydrochloride; toremifene citrate; trestolone acetate; triciribine phosphate; trimetrexate; trimetrexate glucuronate; triptorelin; tubulozole hydrochloride; uracil mustard; uredepa; vapreotide; verteporfin; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-cancer agents under development include:

20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer.

The invention will be more fully understood by reference to the following examples. These examples, however, are merely intended to illustrate the embodiments of the invention and are not to be construed to limit the scope of the invention.

### EXAMPLES

Experimental Procedures

Mice

BALB/c strain of mice used in these experiments were obtained from The Jackson Laboratory (Bar Harbor, Me.). Animals were age matched between 4 and 6 weeks. The animal facility is accredited by the American Association for the Accreditation of Laboratory Animal Care; all procedures were approved by the Institutional Animal Care and Use Committee.

Cell Depletion

Thymocytes were treated in vitro with anti-CD3, 145.2C11, for 30 min, washed with PBS then incubated with baby rabbit complement at 37° C. to deplete CD3⁺, mature TCRαβ+thymocytes.

Antibodies, Staining and Flow Cytometry

Thymocytes were isolated and stained on ice for 30 min, with a rat IgG FITC-anti-mouse CD40, 1C10 (generous gift of M. Howard, DNAX Corp., Palo Alto, Calif.), then washed multiple times with PBS. Fc receptor blocking antibodies (Pharmingen, San Diego, Calif.) were added prior to staining. Cells then were incubated on ice for 30 min with Cy-chrome [fluoresces in FL3] conjugated anti-CD4 (GK1.5) and incubated with phycoerytherin [fluoresces in FL2] conjugated anti-CD8 (both from Pharmingen) and washed. Rat IgG isotype and secondary antibody controls (Pharmingen) were always included. Four color flow cytometic analysis was performed on a FACScalibur™ (Becton Dickinson, San Jose, Calif.). Thymocytes were preincubated with an anti-murine CD32/CD16 F_{c} receptor blocking antibody (2.4G2). Cells were then stained with phycoerytherin conjugated anti-CD8, cychrome conjugated anti-CD4, FITC conjugated anti-TCRαβ (H57-597) and biotin conjugated anti-CD69 (Pharmingen) on ice, for 30 mins. Cells were washed in PBS and incubated with allophycocyanin conjugated streptavidin (Pharmingen). Cells were washed an additional two times in PBS prior to analysis. The FACScalibur, flow cytometer was calibrated using Becton-Dickinson calibration beads prior to each four color analysis run. Cells also were analyzed on a FACScan™ (Becton Dickinson) with CellQuest software (Becton Dickinson). Other antibodies included FITC conjugated TCRVα8 and FITC conjugated TCRVα11 (Pharmingen) and FITC conjugated H57-597. All staining antibodies were used as a 1:100 dilution in PBS.

Induction of TCR

Thymocytes were isolated and treated in vitro with an IgM anti-CD40 (Pharmingen), biotin conjugated 1C10 followed by streptavidin, or a trimeric-CD40L fusion protein (a kind gift of Dr. Richard Armitage, Immunex Corp., Seattle, Wash. (Fanslow W C, et al., Journal of Immunology, 1994, 152: 4262-4269; Fanslow W C, et al., Seminars in Immunology, 1994, 6:267). Cells were incubated 18 h in RPMI, 5% FBS, at 37° C. then stained for CD4/CD8/TCR as previously described and analyzed by flow cytometry.

Results

Expression of CD40 on Thymocytes

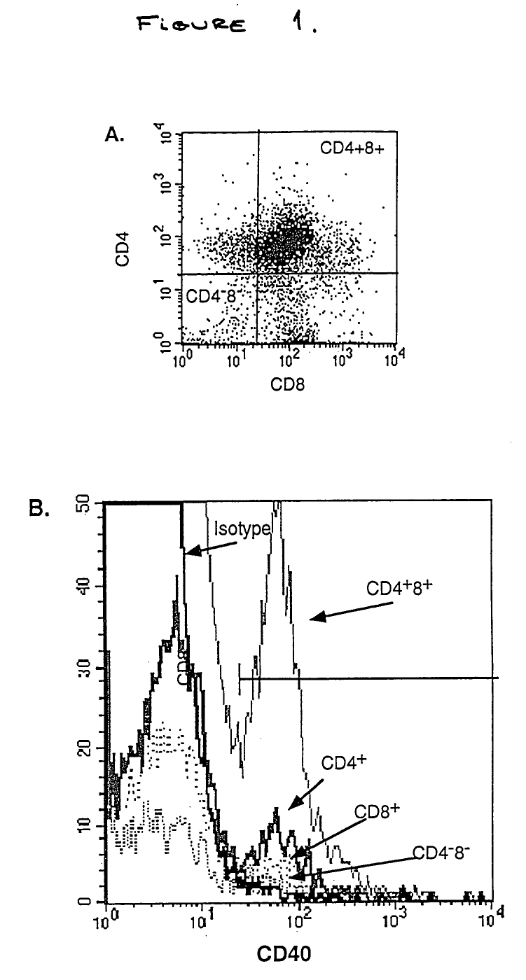

An essential role for CD40/CD40L (CD 154) interactions during thymocyte development has been suggested (Foy T M, et al., J Exp Med, 1995, 182(5):1377-1388). However, in these reports, indirect signalling through CD40L on thymocytes was thought to induce the developmental changes. CD40L typically is expressed on activated peripheral T cells. We considered an alternative possibility; that CD40 may be present on thymocytes and that direct signalling through CD40 induces thymic maturation. Thymocytes from BALB/c mice, as well as numerous other strains, were stained for surface expression of CD40. Thymocyte sub-populations were stained for CD4 and CD8 (FIG. 1A) and CD40 expression levels were determined within each gated sub-population. The CD4⁺8⁺ (DP), and CD4⁺ and CD8⁺ single positive (SP) thymocytes (FIG. 1B—light solid line) expressed detectable levels of CD40 above appropriate isotype controls (FIG. 1B—dark solid line). Levels of expression were relatively consistent on all sub-populations, with a greater number of CD4⁺8⁺ thymocytes expressing CD40 (FIG. 1B—light solid line). Cells were gated on forward light scatter (FSC+) to remove dead and dying cells from the analysis (Wagner D H, Jr., et al., J Exp Med, 1996, 184:1631-1637).

Increased Expression of Mature TCRαβ

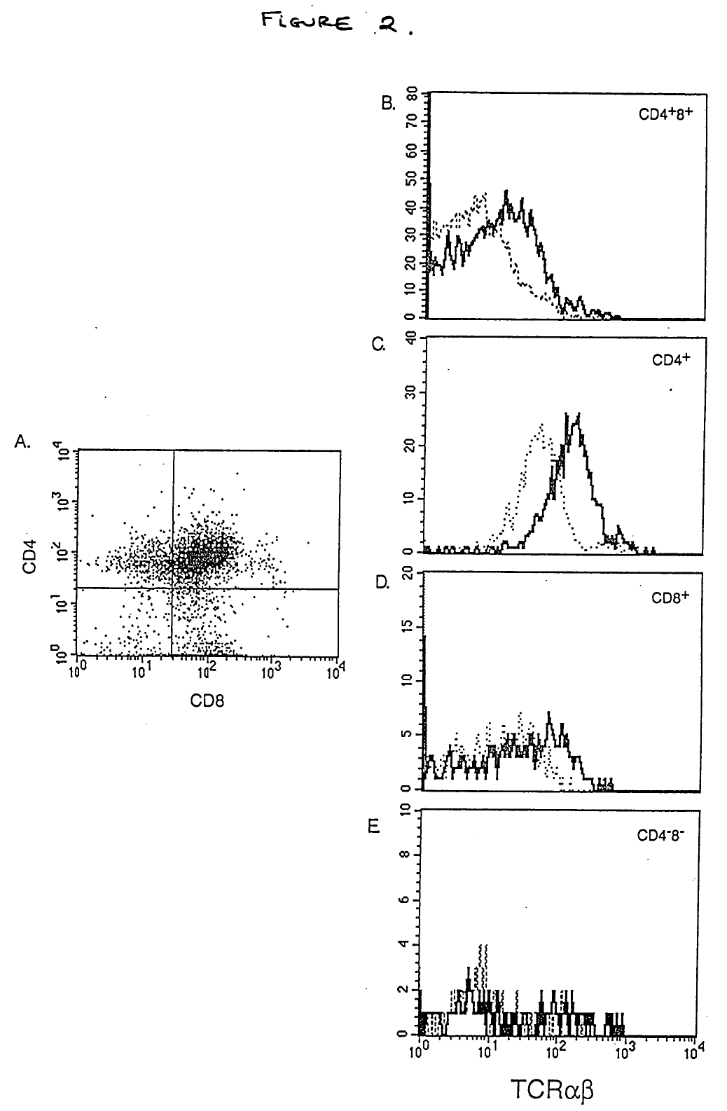

Immature thymocytes express rearranged TCRβ molecules associated with a pre-TCRα molecule (Petrie H T, et al., J Exp Med, 1993, 178:615-622). As thymocytes mature, TCRα rearrangement occurs and the thymocytes progressively express the mature TCRα chain protein (still associated with the rearranged TCRβ) resulting in increased expression of mature TCRαβ. If ligation of CD40 results in rearrangement of the TCRVα gene, then CD40 ligation would lead to increased expression of mature TCRαβ molecules. Because mature thymocytes concomitantly express high levels of CD3 and mature TCRαβ molecules, we depleted the CD3 high, mature TCRαβ⁺ population. After removal of the mature TCRαβ⁺ thymocytes, the remaining thymocytes are TCR⁻ or TCR^{lo}, and therefore CD40 induced TCRVα rearrangement and subsequent expression of TCRαβ on these thymocytes, should be more easily detected. CD4/CD8 staining profiles of partially depleted thymocytes, following overnight incubation, demonstrated that CD4⁺ and CD8⁺ SP populations were generated (FIG. 2A). Partially CD3 depleted thymocytes expressed low levels of TCR in the CD4⁺8⁺ population after overnight culture (FIG. 2B). CD40 crosslinking increased TCRαβ expression in the DP thymocyte population to levels [intermediate/high] (FIG. 2B), equivalent to that seen on untreated CD4⁺, SP thymocytes (FIG. 2C). CD40 crosslinking also induced TCRαβ expression to higher levels on CD4⁺ (FIG. 2C) and CD8⁺ (FIG. 2D). Since the experiments were performed in vitro on CD3 depleted cells, the CD4 committed and CD8 committed cells after overnight culture, likely expressed intermediate levels of TCRαβ (FIGS. 2C and 2D). CD40 signalling induced the cells to TCR^{hi}, more mature levels. Consistent with this explanation is the observation that CD40 crosslinking on CD4⁺8⁺ thymocytes also resulted in a small population that was TCRαβ^{hi} (FIGS. 2B and 2F). Results further were confirmed using a trimeric construct of CD40L fused to a leucine zipper protein (Fanslow W C, et al, J Exp Med, 1994, 152:4262-4269). Crosslinking of CD40 on CD3 depleted thymocytes using the CD40L fusion protein induced increased expression of TCRαβ (FIG. 2F) on CD4⁺8⁺ thymocytes to TCRαβint/hi mature levels (FIG. 2F). CD40 crosslinking had no detectable effect on TCRαβ levels in the DN population (FIG. 2E). Thymocytes were gated on forward light scatter, so that dead cells were excluded from the analysis. Quantitation of cell recovery and assays for apoptosis/cell death (Wagner D H, Jr., et al., J Exp Med, 1996, 184:1631-1637), demonstrated that thymocytes were not induced to die following either antibody or fusion protein treatment.

CD40 Signals Induce Increased Expression of Vα8 but Decreased Expression of Vα11 on Thymocyte CD4⁺8⁺ Populations

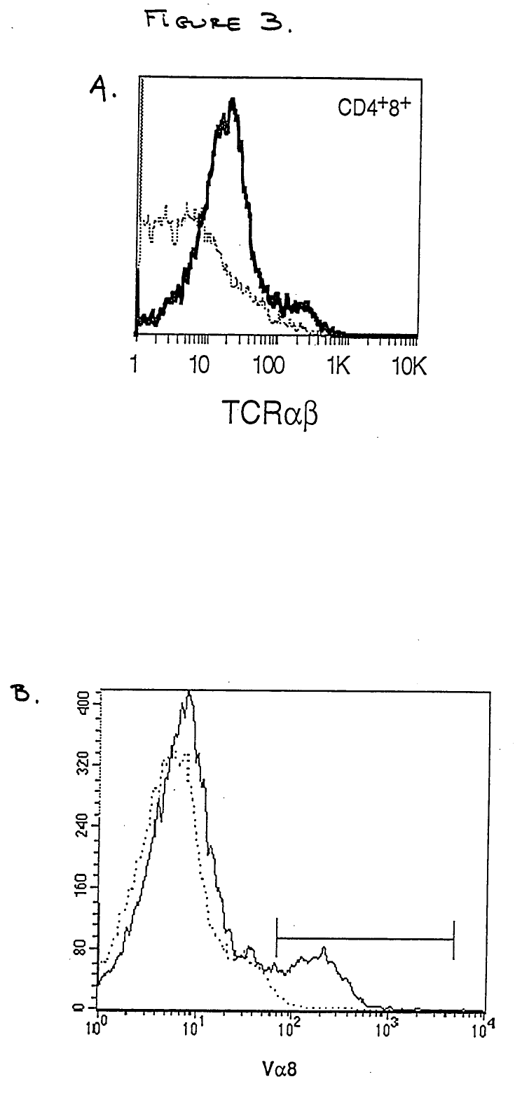

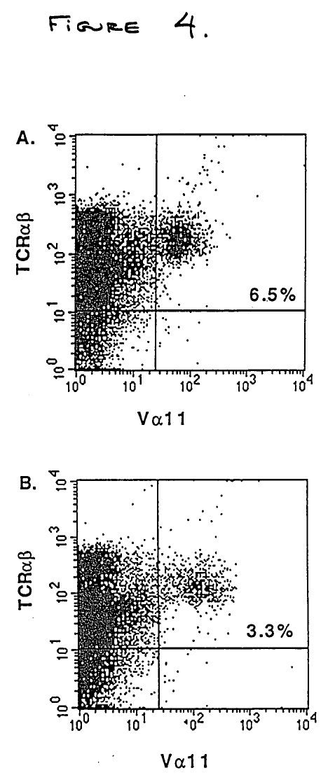

Thymocytes from BALB/c mice were either untreated or treated with biotinylated anti-CD40 (1C10) followed by streptavidin to crosslink. After overnight incubation, cells were stained with directly conjugated antibodies for TCRαβ (FIG. 3A) and TCRVα8 (FIG. 3B) or TCRVα11 (FIG. 4). TCRαβ staining revealed typical thymocyte profiles including TCRαβ^{hi}, TCRαβ^{int} and TCRαβ^{lo} populations. CD40 crosslinking induced a substantial (12%) increase in Vα8 expression in gated DP thymocytes (FIG. 3B). However, CD40 crosslinking caused a reduction in Vα11 expressing thymocytes (FIG. 4A vs. 4B). Vα11⁺ thymocytes were located predominantly within the TCR^{hi} population (FIG. 4A). Untreated thymocytes were 6.5% Vα11⁺ while CD40 crosslinked thymocytes were reduced to 3.3% Vα11⁺ (FIGS. 4A and 4B). This suggests that CD40 crosslinking induces rearrangement of TCRVα genes such that thymocytes expressing TCRVα11 were induced to rearrange the Vα gene and subsequently express some other Vα molecule. The demonstration that the thymocytes are still TCRαβ^{hi}, supports this hypothesis. These data are representative of at least four separate experiments. As before, there were no increases or decreases in total cell numbers between treated and untreated thymocyte cultures, demonstrating that CD40 crosslinking does not induce proliferation of TCRVα8⁺ thymocytes or cell death of TCRVα11⁺ thymocytes.

CD40 Signals Induce Increased Expression of TCRαβ and CD69 on CD4^{lo}8^{lo} Thymocytes

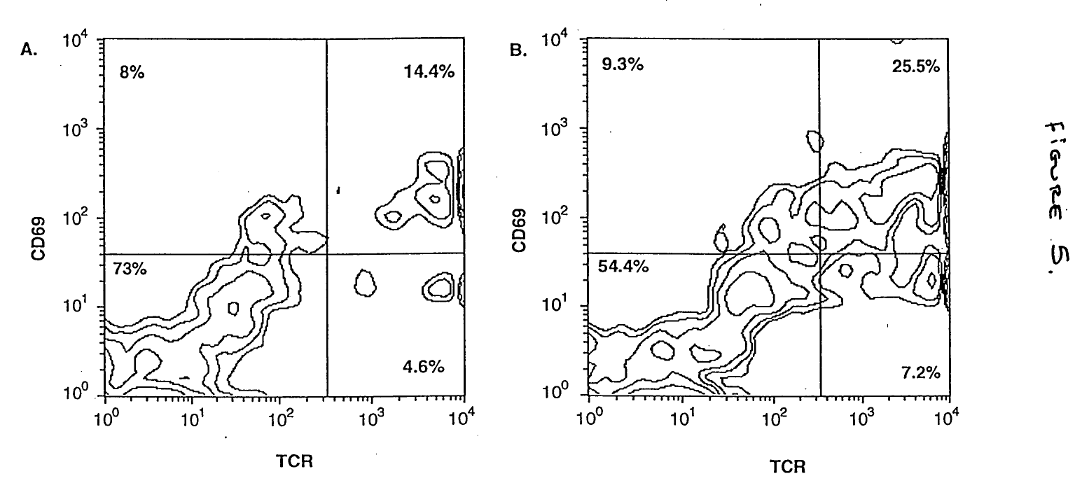

CD4^{lo}8^{lo}CD69⁺TCRαβ⁺ has been suggested to be a more mature developmental stage, potentially even the stage at which thymocytes commit to the CD4 or CD8 SP lineage, during thymocyte maturation (Lucas, B and Germain, R N, Immunity, 1996, 5: 461-477). Four color staining of untreated and anti-CD40 treated thymocytes (anti-TCRαβ-FITC, anti-CD8-phycoerythrin, anti-CD4-Cychrome, and anti-CD69-biotin, followed by APC-streptavidin) was performed. The data demonstrate that CD40 signalling induces a relative increase in the percentage of CD4^{lo}CD8^{lo} cells that co-express CD69 and TCRαβ from 14.4% in untreated (FIG. 5A) to 25.5% in anti-CD40 treated thymocytes (FIG. 5B). CD69 has been defined as a maturational marker for thymocytes (Vanhecke D, et al., Journal of Immunology, 1995, 155:4711-4718). Thus, CD40 ligation induced progression in thymocyte maturation.

Those skilled in the art will recognize, or be able to ascertain using no more than routine experimentation, many equivalents to the specific embodiments of the invention described herein. Such equivalents are intended to be encompassed by the following claims.

All references disclosed herein are incorporated by reference in their entirety.

What is claimed is presented below and is followed by a Sequence Listing.

1. A method for inducing T cell receptor gene rearrangement, comprising:
contacting a T cell with a CD40-binding agent that binds CD40 in an amount sufficient to induce T cell receptor gene rearrangement in the T cell.

2. The method of claim 1, wherein the T cell is present in a lymphocyte population enriched for T cells.

3. The method of claim 2, wherein the lymphocyte population enriched for T cells is furhter enriched for T cells by selectively eliminating B cells.

4. The method of claim 2, wherein the lymphocyte population enriched for T cells contains at least 50% T cells.

5. The method of claim 2, wherein the lymphocyte population enriched for T cells contains at least 75% T cells.

6. The method of claim 2, wherein the lymphocyte population enriched for T cells contains at least 90% T cells.

7. The method of claim 2, wherein the lymphocyte population enriched for T cells contains at least 95% T cells.

8. The method of claim 1, wherein contacting of the T cell with a CD40-binding agent that binds CD40 occurs in vitro.

9. The method of claim 1, wherein contacting of the T cell with a CD40-binding agent that binds CD40 occurs ex vivo.

10. The method of claim 1, wherein the T cell is derived from an in vitro culture of hematopoietic cells.

11. The method of claim 1, wherein the CD40-binding agent comprises at least two agents:
i) a first agent that binds a first CD40 receptor, and
ii) a second crosslinking agent that crosslinks the first agent to at least a second receptor selected from the group consisting of a second CD40 receptor and a T cell receptor.

12. The method of claim 11, wherein the first agent that binds a first CD40 receptor is selected from the group consisting of a CD40 ligand and an anti-CD40 antibody.

13. The method of claim 11, wherein the second crosslinking agent that crosslinks the first agent to the second receptor is selected from the group consisting of a CD40 ligand, an anti-CD40 antibody and an antigen.

14. The method of claim 12, wherein the CD40 ligand is the polypeptide of SEQ ID NO:2 or a fragment thereof.

15. The method of claim 13, wherein the CD40 ligand is the polypeptide of SEQ ID NO:2 or a fragment thereof.

16. The method of claim 1, wherein the T cell is of a CD69⁺TCR⁺ phenotype.

17. The method of claim 1, wherein the T cell is of a phenotype selected from the group consisting of CD4^{lo}CD8^{lo}CD69⁺TCR⁺, CD4^{lo}CD8^{hi}CD69⁺TCR⁺, and CD4^{hi}CD8^{lo}CD69⁺TCR⁺.

18. The method of claims 1-17, further comprising administering a co-stimulator agent, wherein the co-stimulatory agent is selected from the group consisting of a co-stimulatory molecule and a cytokine.

19. The method of claim 18, wherein the co-stimulatory molecule is selected from the group consisting of TSA-1, CD5CD5, CD24, CD28, CD49a, CD80, CD81 and CD86.

20. The method of claim 18, wherein the cytokine is selected from the group consisting of IL-2 and IL-4.

21. A method for promoting T cell maturation, comprising:
contacting an immature T cell with a CD40-binding agent that binds CD40 in an amount sufficient to promote maturation of the immature T cell.

22. The method of claim 21, wherein the T cell is present in a lymphocyte population enriched for T cells.

23. The method of claim 22, wherein the lymphocyte population enriched for T cells is further enriched for T cells by selectively eliminating B cells.

24. The method of claim 22, wherein the lymphocyte population enriched for T cells contains at least 50% T cells.

25. The method of claim 22, wherein the lymphocyte population enriched for T cells contains at least 75% T cells.

26. The method of claim 22, wherein the lymphocyte population enriched for T cells contains at least 90% T cells.

27. The method of claim 22, wherein the lymphocyte population enriched for T cells contains at least 95% T cells.

28. The method of claim 21, wherein contacting of the T cell with a CD40-binding agent that binds CD40 occurs in vitro.

29. The method of claim 21, wherein contacting of the T cell with a CD40-binding agent that binds CD40 occurs ex vivo.

30. The method of claim 21, wherein the T cell is derived from an in vitro culture of hematopoietic cells.

31. The method of claim 21, wherein the CD40-binding agent comprises at least two agents:
i) a first agent that binds a first CD40 receptor, and
ii) a second crosslinking agent that crosslinks the first agent to at least a second receptor selected from the group consisting of a second CD40 receptor and a T cell receptor.

32. The method of claim 31, wherein the first agent that binds a first CD40 receptor is selected from the group consisting of a CD40 ligand and an anti-CD40 antibody.

33. The method of claim 31, wherein the second crosslinking agent that crosslinks the first agent to the second receptor is selected from the group consisting of a CD40 ligand, an anti-CD40 antibody and an antigen.

34. The method of claim 32, wherein the CD40 ligand is the polypeptide of SEQ ID NO:2 or a fragment thereof.

35. The method of claim 33, wherein the CD40 ligand is the polypeptide of SEQ ID NO:2 or a fragment thereof.

36. The method of claim 21, wherein the T cell is of a CD69⁻TCR^{lo} phenotype.

37. The method of claim 21, wherein the T cell is of a phenotype selected from the group consisting of CD4⁺CD8⁺TCR^{lo}, and CD¹¹⁷⁺TCR^{lo}.

38. The method of claims 21-37, further comprising administering a co-stimulatory agent, wherein the co-stimulatory agent is selected from the group consisting of a co-stimulatory molecule and a cytokine.

39. The method of claim 38, wherein the co-stimulatory molecule is selected from the group consisting of TSA-1, CD2, CD5, CD24, CD28, CD49a, CD80, CD81 and CD86.

40. The method of claim 38, wherein the cytokine is selected from the group consisting of IL-2 and IL-4.

41. A method for inhibiting T cell receptor gene rearrangement, comprising:
contacting a T cell expressing CD40 with an agent that inhibits CD40-induced T cell receptor rearrangement.

42. The method of claim 41, wherein contacting of the T cell expressing CD40 with an agent that inhibits CD40-induced T cell receptor rearrangement occurs in vitro.

43. The method of claim 41, wherein contacting of the T cell expressing CD40 with an agent that inhibits CD40-induced T cell receptor rearrangement occurs ex vivo.

44. The method of claim 41, wherein the T cell is derived from an in vitro culture of hematopoietic cells.

46. The method of claim 41, wherein the agent that inhibits CD40-induced T cell receptor rearrangement is selected from the group consisting of an anti-CD40 ligand antibody, a soluble CD40 ligand antagonist, and a NF-κB inhibitor.

47. A method for inducing T cell reactivity toward an antigen, comprising:
introducing an amount of T cells and an amount of antigen presenting cells into a culture vessel, and
co-culturing the T cells and the antigen presenting cells in the presence of:
(i) a CD40-binding agent that binds CD40 in an amount sufficient to induce T cell receptor gene rearrangement in the T cells, and
(ii) at least one antigen,
under conditions sufficient to induce the formation of T cells having specificity for the at least one antigen.

48. The method of claim 47, wherein the T cell is present in a lymphocyte population enriched for T cells.

49. The method of claim 48, wherein the lymphocyte population enriched for T cells is further enriched for T cells by selectively eliminating B cells.

50. The method of claim 48, wherein the lymphocyte population enriched for T cells contains at least 50% T cells.

51. The method of claim 48, wherein the lymphocyte population enriched for T cells contains at least 75% T cells.

52. The method of claim 48, wherein the lymphocyte population enriched for T cells contains at least 90% T cells.

53. The method of claim 48, wherein the lymphocyte population enriched for T cells contains at least 95% T cells.

54. The method of claim 47, wherein contacting of the T cell with a CD40-binding agent that binds CD40 occurs in vitro.

55. The method of claim 47, wherein contacting of the T cell with a CD40-binding agent that binds CD40 occurs ex vivo.

56. The method of claim 47, wherein the T cell is derived from an in vitro culture of hematopoietic cells.

57. The method of claim 47, wherein the CD40-binding agent comprises at least two agents:
i) a first agent that binds a first CD40 receptor, and
ii) a second crosslinking agent that crosslinks the first agent to at least a second receptor selected from the group consisting of a second CD40 receptor and a T cell receptor.

58. The method of claim 57, wherein the first agent that binds a first CD40 receptor is selected from the group consisting of a CD40 ligand and an anti-CD40 antibody.

59. The method of claim 57, wherein the second crosslinking agent that crosslinks the first agent to the second receptor is selected from the group consisting of a CD40 ligand, an anti-CD40 antibody and an antigen.

60. The method of claim 58, wherein the CD40 ligand is the polypeptide of SEQ ID NO:2 or a fragment thereof.

61. The method of claim 59, wherein the CD40 ligand is the polypeptide of SEQ ID NO:2 or a fragment thereof.

62. The method of claim 47, wherein the T cell is of a CD69⁺TCR⁺ phenotype.

63. The method of claim 47, wherein the T cell is of a phenotype selected from the group consisting of CD4^{lo}CD8^{lo}CD69⁺TCR⁺, CD4^{lo}CD8^{hi}CD69⁺TCR⁺, and CD4^{hi}CD8^{lo}CD69⁺TCR⁺.

64. The method of claims 47-63, further comprising administering a co-stimulatory agent, wherein the co-stimulatory agent is selected from the group consisting of a co- stimulatory molecule and a cytokine.

65. The method of claim 64, wherein the co-stimulatory molecule is selected from the group consisting of TSA-1, CD2, CD5, CD24, CD28, CD49a, CD80, CD81 and CD86.

66. The method of claim 64, wherein the cytokine is selected from the group consisting of IL-2 and IL-4.

67. A method for inhibiting environmental stress-induced cell-death of a T cell, comprising:
contacting a T cell expressing CD40, under environmental stress otherwise sufficient to induce cell-death, with a CD40-binding agent that binds CD40 and induces T cell receptor gene rearrangement in an amount sufficient to inhibit death of the cell expressing CD40 which otherwise would result from the environmental stress.

68. The method of claim 67, wherein the T cell is present in a lymphocyte population enriched for T cells.

69. The method of claim 68, wherein the lymphocyte population enriched for T cells is further enriched for T cells by selectively eliminating B cells.

70. The method of claim 68, wherein the lymphocyte population enriched for T cells contains at least 50% T cells.

71. The method of claim 68, wherein the lymphocyte population enriched for T cells contains at least 75% T cells.

72. The method of claim 68, wherein the lymphocyte population enriched for T cells contains at least 90% T cells.

73. The method of claim 68, wherein the lymphocyte population enriched for T cells contains at least 95% T cells.

74. The method of claim 67, wherein contacting of the T cell with a CD40-binding agent that binds CD40 occurs in vitro.

75. The method of claim 67, wherein contacting of the T cell with a CD40-binding agent that binds CD40 occurs ex vivo.

76. The method of claim 67, wherein the T cell is derived from an in vitro culture of hematopoietic cells.

77. The method of claim 67, wherein the CD40-binding agent comprises at least two agents:
i) a first agent that binds a first CD40 receptor, and
ii) second crosslinking agent that crosslinks the first agent to at least a second receptor selected from the group consisting of a second CD40 receptor and a T cell receptor.

78. The method of claim 77, wherein the first agent that binds a first CD40 receptor is selected from the group consisting of a CD40 ligand and an anti-CD40 antibody.

79. The method of claim 77, wherein the second crosslinking agent that crosslinks the first agent to the second receptor is selected from the group consisting of a CD40 ligand, an anti-CD40 antibody and an antigen.

80. The method of claim 78, wherein the CD40 ligand is the polypeptide of SEQ ID NO:2 or a fragment thereof.

81. The method of claim 79, wherein the CD40 ligand is the polypeptide of SEQ ID NO:2 or a fragment thereof.

82. The method of claim 67, wherein the T cell is of a phenotype selected from the group consisting of CD69⁺TCR⁺, CD4^{lo}CD8^{lo}CD69⁺TCR⁺, CD4^{lo}CD8^{hi}CD69⁺TCR⁺, and CD4^{hi}CD8^{lo}CD69⁺TCR⁺.

83. The method of claim 67, wherein the T cell is of a phenotype selected from the group consisting of CD69⁻TCR^{lo}, CD4⁺CD8⁺TCR^{lo}, and CD117⁺TCR^{lo}.

84. The method of claim 67, wherein the environmental stress is selected from the group consisting of chemical stress, physical stress, oxidative stress, and γ-irradiation.

85. A method for enhancing environmental stress-induced T cell-death, comprising:
contacting a T cell expressing CD40 with a CD40-binding agent that binds CD40 in an amount sufficient to induce T cell receptor gene rearrangement,
subjecting the CD40-binding agent bound T cell to an environmental stress sufficient to induce cell-death.

86. The method of claim 85, wherein the T cell is present in a lymphocyte population enriched for T cells.

87. The method of claim 86, wherein the lymphocyte population enriched for T cells is further enriched for T cells by selectively eliminating B cells.

88. The method of claim 86, wherein the lymphocyte population enriched for T cells contains at least 50% T cells.

89. The method of claim 86, wherein the lymphocyte population enriched for T cells contains at least 75% T cells.

90. The method of claim 86, wherein the lymphocyte population enriched for T cells contains at least 90% T cells.

91. The method of claim 86, wherein the lymphocyte population enriched for T cells contains at least 95% T cells.

92. The method of claim 85, wherein contacting of the T cell with a CD40-binding agent that binds CD40 occurs in vitro.

93. The method of claim 85, wherein contacting of the T cell with a CD40-binding agent that binds CD40 occurs ex vivo.

94. The method of claim 85, wherein the T cell is derived from an in vitro culture of hematopoietic cells.

95. The method of claim 85, wherein the CD40-binding agent comprises at least two agents:
i) a first agent that binds a first CD40 receptor, and
ii) a second crosslinking agent that crosslinks the first agent to at least a second receptor selected from the group consisting of a second CD40 receptor and a T cell receptor.

96. The method of claim 95, wherein the first agent that binds a first CD40 receptor is selected from the group consisting of a CD40 ligand and an anti-CD40 antibody.

97. The method of claim 95, wherein the second crosslinking agent that crosslinks the first agent to the second receptor is selected from the group consisting of a CD40 ligand, an anti-CD40 antibody and an antigen.

98. The method of claim 96, wherein the CD40 ligand is the polypeptide of SEQ ID NO:2 or a fragment thereof.

99. The method of claim 97, wherein the CD40 ligand is the polypeptide of SEQ ID NO:2 or a fragment thereof.

100. The method of claim 85, wherein the T cell is of a phenotype selected from the group consisting of CD69⁺TCR⁺, CD4^{lo}CD8^{lo}CD69⁺TCR⁺, CD4^{lo}CD8^{hi}CD69⁺TCR⁺, and CD4^{hi}CD8^{lo}CD69⁺TCR⁺.

101. The method of claim 85, wherein the T cell is of a phenotype selected from the group consisting of CD69⁻TCR^{lo}, CD4⁺CD8⁺TCR^{lo}, and CD117⁺TCR^{lo}.

102. The method of claim 85, wherein the environmental stress is selected from the group consisting of chemical stress, physical stress, oxidative stress, and γ-irradiation.

103. The method of claim 85, wherein the T cell is selected from the group consisting of a cancerous T cell and a self-reactive T cell, and the environmental stress is a chemotherapeutic agent.


SEQUENCE LISTING (2 sequences)

SEQ ID NO: 1 - DNA, 1816 bp, Homo Sapiens
cttctctgccagaagataccatttcaactttaacacagcatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctgaacagtgtcaccttgcaggctgtggtggagctgacgctgggagtcttcataatacagcacagcggttaagcccaccccctgttaactgcctatttataaccctaggatcctccttatggagaactatttattatacactccaaggcatgtagaactgtaataagtgaattacaggtcacatgaaaccaaaacgggccctgctccataagagcttatatatctgaagcagcaaccccactgatgcagacatccagagagtcctatgaaaagacaaggccattatgcacaggttgaattctgagtaaacagcagataacttgccaagttcagttttgtttctttgcgtgcagtgtctttccatggataatgcatttgatttatcagtgaagatgcagaagggaaatggggagcctcagctcacattcagttatggttgactctgggttcctatggccttgttggagggggccaggctctagaacgtctaacacagtggagaaccgaaacccccccccccccccccgccaccctctcggacagttattcattctctttcaatctctctctctccatctctctctttcagtctctctctctcaacctctttcttccaatctctctttctcaatctctctgtttccctttgtcagtctcttccctcccccagtctctcttctcaatccccctttctaacacacacacacacacacacacacacacacacacacacacacacacacacacacacacacacacagagtcaggccgttgctagtcagttctcttctttccaccctgtccctatctctaccactatagatgagggtgaggagtagggagtgcagccctgagcctgcccactcctcattacgaaatgactgtatttaaaggaaatctattgtatctacctgcagtctccattgtttccagagtgaacttgtaattatcttgttatttattttttgaataataaagacctcttaacattaaaa

SEQ ID NO: 2 - protein, 261 aa, Homo Sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL